(19) United States
(12) Patent Application Publication
(10) Pub. No.: US 2019/0135934 A1
(43) Pub. Date: May 9, 2019

(54) ANTI-CD20 TARGETED ANTIBODY, AND USES AND TECHNICAL FIELD

(71) Applicants: Lei ZHAO, Beijing (US); Fan ZHANG, Beijing (US)

(72) Inventors: Lei ZHAO, Beijing (US); Fan ZHANG, Beijing (US)

(21) Appl. No.: 16/095,968

(22) PCT Filed: Jun. 13, 2016
(86) PCT No.: PCT/CN2016/085531

§ 371 Date: Oct. 24, 2018

(30) Foreign Application Priority Data
Apr. 24, 2016 (CN) 201610257652.2

(51) Int. Cl. C07K 16/28; A61K 45/06; A61K 39/395; A61P 35/00

(52) U.S. Cl. CPC C07K 16/2887; A61K 45/06; A61K 39/3955; A61K 2039/505; C07K 2317/734; C07K 2317/24; C07K 2317/732; A61P 35/00

(57) ABSTRACT

Provided are a CD20 antibody having advantages of both a type-I CD20 antibody and a type II CD20 antibody, and nucleotide and amino acid sequences thereof, by which cells can be induced to die and anti-tumor drugs can be prepared

### TECHNICAL FIELD

The present invention relates to the field of biotechnology, and more particularly, the present invention discloses a class of anti-CD20 targeted antibodies, the preparation method and the use in the preparation of anti-tumor antibody drugs thereof. The anti-CD20 targeted antibody disclosed in the present invention not only has the anti-tumor killing advantage of type I CD20 antibody, which induces CDC to kill tumor cells, but also has the anti-tumor advantage of the type II CD20 antibody, which can induce strong cell death to kill tumor cells. The antibody exhibits better anti-tumor activity than the type I CD20 antibody Rituximab.

### BACKGROUND

Leukemia of non-Hodgkin's lymphoma (NHL) is the most common hematological and lymphatic malignancy in the clinic and often occurs in young adults. The morbidity and the mortality of NHL are increasing year by year, 85% of which are originated from B cells. CD20 antigen is a B cell differentiation antigen located only in pre-B cells, immature B cells and mature B cells, which is expressed in more than 95% of B-cell lymphomas, but not in hematopoietic stem cells, plasma cells, or other normal tissue cells. More importantly, the CD20 molecule is a four-pass transmembrane protein on the membrane, which has no significant endocytosis or shedding after binding to the targeted antibody, and does not undergo antigen modulation after antibody ligation. Therefore, CD20 is an ideal target for the treatment of B-cell lymphoma.

CD20 monoclonal antibody therapy has achieved satisfactory results in the treatment of B cell lymphoma. The CD20 antibody Rituximab has been used as a first-line drug and is widely used in the clinical treatment of B cell lymphoma. Rituximab has made a breakthrough in the clinical administration of B cell lymphoma, which has nearly 50% response rates and about 10% complete response rates, and could be considered as one of the most effective tumor-targeted antibody drugs. However, most patients would gradually develop recurrence and resistance during the treatment. Therefore, there is an urgent need to design of CD20 antibodies with more potent anti-tumor efficacy against B cell lymphoma.

It is currently believed that the anti-tumor mechanisms of CD20-targeted antibodies include: complement-dependent cytotoxicity (CDC), antibody-dependent cell-mediated cytotoxicity (ADCC), and induction of tumor cell death (Cell Death). According to the mechanisms of action of CD20 targeted antibodies, the antibodies can be divided into two types: type I CD20 antibodies, including Rituximab and most CD20 antibodies (1F5, 2H7, 2F2, etc.), which can induce strong CDC killing but weak cell death; type II CD20 antibodies, including B1, 11B8 and Obinutuzumab, which are capable of inducing strong cell death but weak CDC killing. The CD20 antibodies approved for clinical treatment are mainly type I CD20 antibodies (Rituximab and 2F2), with the exception of the conjugated drug of type II antibody B1 and the 13I Tositumomab and the recently approved Obinutuzumab. Type I CD20 antibodies have achieved good therapeutic effects in the clinical treatment of B cell lymphoma, indicating that strong CDC killing induced by type I CD20 antibodies plays a very important role in the clinical treatment of B lymphoma. However, as the research progressed, the important role of strong caspase-independent cell death induced by type II CD20 antibody gradually emerged in the treatment of lymphoma. The type II CD20 antibody Obinutuzumab shows good tumor killing effects in both preclinical and clinical trials. Although Obinutuzumab is a glycosylation-modified type II CD20 antibody, further studies have instructed that Obinutuzumab with glycosylation to enhance its ADCC and antibody-dependent cellular phagocytosis (ADCP) has no significant difference from non-glycosylation-modified Obinutuzumab in the clearance of B cells in vivo of mice. More importantly, the type II CD20 antibody Obinutuzumab showed better therapeutic effects than the type I CD20 antibody Rituximab in clinical trials of lymphoma, indicating that strong cell death induced by type II CD20 antibody plays more important role in the treatment of B lymphoma. Currently, the US FDA has approved Obinutuzumab for clinical treatment of chronic lymphocytic leukemia. These findings not only indicate that both CDC killing and cell death induced by CD20 targeted antibody play a very important role in effective killing of lymphoma cells by targeted antibody, but also reveal that caspase-independent cell death induced by CD20-targeted antibody is more important in the killing of lymphoma. However, at present, no such novel CD20 antibody having the advantages of both type I and type II CD20 antibodies has been reported. Therefore, how to design a novel CD20 antibody having the advantages of both type I and type II CD20 antibodies, which can simultaneously induce strong CDC killing and cell death is a hotspot and a difficult point in the field of targeted antibody research at present.

### SUMMARY OF THE INVENTION

In order to solve the above problems, the inventors of the present invention conducted long-term research and lots of experiments, using genetic engineering technologies, constructed novel CD20 antibodies with the advantages of both type I and type II CD20 antibodies could induce potent CDC and cell death. By introducing a single point mutation in the CDR of type I CD20 antibody Rituximab, the antibody obtained the capacity to initiate marked CDC and cell death, showing better anti-tumor activity than the type I CD20 antibody Rituximab.

This invention disclosed that:

1. A novel CD20 antibodies with the anti-tumor advantages of both type I and type II CD20 antibodies have the capacity to induce strong CDC and cell death against CD20-positive B lymphoma cells.

2. The aforementioned novel CD20 antibody, consisting of four peptide chains, which are two mutants of Rituximab heavy chain and two Rituximab Light chains, respectively.

3. An isolated nucleotide molecule encoding four peptide chains that form the aforementioned novel CD20 antibody, which are Rituximab heavy chain mutants and Rituximab light chains.

4. A vector comprising the aforementioned novel CD20 antibody or the nucleic acid molecule thereof and an expression regulatory sequence operably linked to the sequence of the nucleic acid molecule, wherein the vector may be one of pDR1, pcDNA3.1(+) or pcDNA3.1/ZEO(+), and pDHFR.

5. The vector is one of pDR1, pcDNA3.1(+), pcDNA3.1/ZEO(+), and pDHFR.

6. A host cell comprising the vector is a eukaryotic cell.

7. The host cell is a mammalian cell.

8. The host cell is a CHO cell.

9. A composition comprising the novel CD20 antibody and a pharmaceutically acceptable carrier.

10. Use of the novel CD20 antibody for the preparation of an antibody tumor drug.

11. Use of the composition for the preparation of an anti-tumor drug.

12. Any of the above use, which is in combination with using other anti-tumor drugs.

The propose of the present disclosure is to provide novel CD20 antibodies that have the anti-tumor advantages of both type I and type II CD20 antibodies. The novel CD20 antibody retains both the advantage of strong CDC killing effect induced by type I CD20 antibody Rituximab and the advantage of type II CD20. The novel CD20 antibodies with the benefit to induce strong cell death exhibit better anti-tumor activity than the single-agent parental antibody Rituximab.

The present invention deeply understands the structure and function of CD20 antibodies, and on the basis of which, designs a novel CD20 antibody which has the advantages of both type I and type II CD20 antibodies. In order to demonstrate that this method has a better therapeutic effect than the single drug CD20 antibody Rituximab, the inventors conducted an experiment of subsequent protein antitumor effects.

In the present invention, any suitable vector can be used, which may be pDR1, pcDNA3.1(+), pcDNA3.1/ZEO(+) or pDHFR et. al. The expression vector contains fusion DNA sequences which are linked to suitable transcriptional and translational regulatory sequences.

A culture system of mammalian or insect host cell can be used for expression of the novel CD20 antibodies provided in the present invention, and cells such as COS, CHO, NSO, sf9 and sf21 can be suitable for use in the present invention.

A suitable host cell is a prokaryotic cell containing the vector, which may be one of DH5a, BL21(DE3), and TG1.

For the novel CD20 antibody disclosed in the present invention, the host cell is cultured in the expression condition thereby expressed, isolated and purified the novel CD20 antibody.

The novel CD20 antibody disclosed in the present invention can be isolated and purified by affinity chromatography. According to the characteristics of the affinity column utilized, conventional methods such as high salt buffer elution, pH change, etc. can be used to elute the novel CD20 antibody binding to the affinity column.

Using the above method, the novel CD20 antibody can be purified to a substantially homogeneous substance, such as a single band on SDS-PAGE electrophoresis.

The novel CD20 antibody or composition can be used in the preparation of an anticancer drug, and also can be used in combination with other antitumor drugs.

The novel CD20 antibody disclosed in the present invention, can form a pharmaceutical formulation by mixing together with a pharmaceutically acceptable excipient to exert a more stable therapeutic effect. These formulations can ensure the conformational integrity of the core amino acid sequence of the novel CD20 antibody disclosed in the present invention, and at the same time protect the multiple functional groups of the protein from degradation (including but not limited to coagulation, deamination or oxidation). Generally, liquid formulations are typically stable for at least one year at 2° C. to 8° C. and lyophilized formulations are stable for at least six months at 30° C. The formulations may be suspension, aqueous injection solution, or lyophilized formulation, etc., which are commonly used in the pharmaceutical field, wherein aqueous solution or lyophilized formulation is preferred. For the aqueous injection solution or lyophilized formulation of the novel CD20 antibody disclosed above, the pharmaceutically acceptable excipient includes one of surfactants, solution stabilizers, isotonicity adjusting agents and buffer solutions, or a combination thereof.

The surfactants include nonionic surfactants such as polyoxyethylene sorbitan fatty acid esters (Tween 20 or 80); poloxamer (such as poloxamer 188); Triton; sodium dodecyl sulfate (SDS); sodium lauryl sulfate; tetradecyl, linoleyl or octadecyl sarcosine; Pluronics; MONAQUATTM, etc., the addition amount of which should minimize the granulation tendency of the bifunctional fusion protein. The solution stabilizer may be a sugar including reducing sugar and non-reducing sugaror, amino acids include monosodium glutamate or histidine, alcohols includeone of a trihydroxy alcohol, a higher sugar alcohol, a propylene glycol, and a polyethylene glycol or a combination thereof. The solution stabilizer is added in an amount such that the final formed formulation remains stable for a period of time that is considered stable by those skilled in the art.

The isoosmotic adjusting agent may be one of sodium chloride and mannitol, and the buffer may be one of TRIS, histidine buffer, and phosphate buffer.

The above formulation is a composition comprising the novel CD20 antibody, and the anti-tumor effect is remarkable after administration of the formulation to animals including human. Specifically, it is effective for the prevention and/or treatment of tumors and can be used as an anti-tumor drug.

When the dual-targeted antibody and its composition in the present invention are administered to animals including human, the dosage is different depending on the age and weight of the patient, the disease characteristics and severity, and the administration route, and which can refer to the results and various conditions of an animal experiment. The total dose can not exceed a certain range. Specifically, the dose for intravenous injection is 0.1 to 3000 mg/day.

The term “anti-tumor drug” as used in the present invention refers to a drug able to inhibit and/or treat tumor, which may include a delay accompanying the development of the symptoms associated with tumor growth and/or a decrease of the symptom severity, and further alleviate and prevent other symptoms of existing tumor growth, and also decrease or prevent tumor metastasis.

The novel CD20 antibodies and compositions thereof disclosed in the present invention can also be administered for the treatment of tumors in combination with other anti-tumor drugs, which include: 1. Cytotoxic drugs (1) Drugs acting on the chemical structure of DNA: alkylating agents such as nitrogen mustards, nitrosoureas, methyl sulfonates; platinum compounds such as cisplatin, carboplatin and Oxaliplatin and the like; mitomycin (MMC); (2) Drugs affecting nucleic acid synthesis: dihydrofolate reductase inhibitors such as methotrexate (MTX) and Alimta, etc; thymidine synthase inhibitors such as fluorouracil (5FU, FT-207, capecitabine), etc.; purine nucleoside synthase inhibitors such as 6-mercaptopurine (6-MP) and 6-TG, etc.; nucleoside reductase inhibitors such as hydroxyurea (HU), etc.; DNA polymerase inhibitors such as cytarabine (Ara-C) and Gemzar, etc.; (3) Drugs that act on nucleic acid transcription: drugs that selectively act on DNA templates and inhibit DNA-dependent RNA polymerase, thereby inhibiting RNA synthesis, such as actinomycin D, daunorubicin, doxorubicin, epirubicin, aclarubicin, mithramycin, etc.; (4) Drugs mainly acting on tubulin synthesis: paclitaxel, taxotere, vinblastine, vinorelbine, podophyllum, homoharringtonine; (5) Other cytotoxic drugs: Asparaginase mainly inhibiting protein synthesis; 2. hormone: (1)anti-estrogen: tamoxifen, droloxifene, exemestane, etc.; (2)aromatase inhibitors: aminoglutethimide, lentaron, letrozole, anastrozole, etc.; anti-androgen: flutamide; RH-LH agonist/antagonist: zoladex, enatone, etc.; 3. biological response modifier: tumor interferon is inhibited mainly through the body's immune function ; interleukin-2; thymosin; 4. monoclonal antibodies: Rituximab (MabThera); Cetuximab (C225); Herceptin (Trastuzumab); Bevacizumab (Avastin); 5. Other drugs that the mechanismis currently unknown and need to be further studied: Cell differentiation inducers such as retinoids ; apoptosis inducers. The dual targeted antibody and the composition thereof disclosed in the present invention can be used in combination with one of the above anti-tumor drugs and a combination thereof.

### DESCRIPTION OF THE DRAWINGS

FIG. 1. Antigen binding activity of novel CD20 antibodies.

FIG. 2. CDC killing activity of novel CD20 antibodies.

FIG. 3. Induction of Cell death by novel CD20 antibodies.

FIG. 4. ADCC killing activity of novel CD20 antibodies.

FIG. 5. in vivo anti-tumor activity of novel CD20 antibodies in mouse model.

### EMBODIMENTS FOR CARRYING OUT THE INVENTION

### Example 1. Construction of Heavy Chain Variable Region and Light Chain Variable Region Genes of Novel CD20 Antibody

Using the Rituximab antibody heavy chain as a template, mutations were introduced into the 102nd position of the heavy chain variable region by overlap PCR, and the amino acid was mutated to His, Leu, Met, Asn, Gln, Arg, Ser, Thr, Val or Trp, separately. The Rituximab heavy chain variable region mutant was then ligated to the antibody constant region and loaded into an expression vector. SEQ ID NO: 1 shows the amino acid sequence of Rituximab antibody heavy chain variable region, the nucleotide sequence of which is SEQ ID NO: 2; SEQ ID NO: 3 shows the amino acid sequence of the Rituximab antibody heavy chain mutant H102YH variable region, the nucleotide sequence of which is SEQ ID NO: 4; SEQ ID NO: 5 shows the amino acid sequence of Rituximab antibody heavy chain mutant H102YL variable region, the nucleotide sequence of which is SEQ ID NO: 6; SEQ ID NO: 7 shows the amino acid sequence of Rituximab antibody heavy chain mutant H102YM variable region, the nucleotide sequence of which is SEQ ID NO: 8; SEQ ID NO: 9 shows the amino acid sequence of the Rituximab antibody heavy chain mutant H102YN variable region, the nucleotide sequence of which is SEQ ID NO: 10; SEQ ID NO: 11 shows the amino acid sequence of the Rituximab antibody heavy chain mutant H102YQ variable region, the nucleotide sequence of which is SEQ ID NO: 12; SEQ ID NO: 13 shows the amino acid sequence of the Rituximab antibody heavy chain mutant H102YR variable region, the nucleotide sequence of which is SEQ ID NO: 14; SEQ ID NO: 15 shows the amino acid sequence of the Rituximab antibody heavy chain mutant H102YS variable region, the nucleotide sequence of which is SEQ ID NO: 16; SEQ ID NO: 17 shows the amino acid sequence of the Rituximab antibody heavy chain mutant H102YT variable region, the nucleotide sequence of which is SEQ ID NO: 18; SEQ ID NO: 19 shows the amino acid sequence of the Rituximab antibody heavy chain mutant H102YV variable region, the nucleotide sequence of which is SEQ ID NO: 20; SEQ ID NO: 21 shows the amino acid sequence of the Rituximab antibody heavy chain mutant H102YW variable region, the nucleotide sequence of which is SEQ ID NO: 22; SEQ ID NO: 23 shows the amino acid sequence of the Rituximab antibody light chain variable region, the nucleotide sequence of which is SEQ ID NO: 24.

### Example 2. Cloning of Antibody Heavy Light Chain Constant Regions

The healthy human lymphocytes were isolated using lymphocyte separation solution (product from Dingguo Biotechnology Development Co., Ltd.) and total RNA was extracted using Trizol reagent (Invitrogen). The antibody heavy chain constant region and light chain constant region genes were amplified by RT-PCR reaction, according to “Cloned human and mouse kappa immunoglobulin constant and J region genes conserve homology in functional segments.” Hieter PA, Max EE, Seidman JG, Maizel J V Jr, Leder P. Cell. 1980 November; 22(1 Pt 1):197-207 and “The nucleotide sequence of a human immunoglobulin C gamma1 gene.” Ellison J W, Berson B J, Hood L E. Nucleic Acids Res. 1982 Jul. 10; 10(13):4071-9. The PCR products were purified and recovered using agarose gel electrophoresis and cloned into pGEM-T vector. After sequencing, the correct clone was identified. SEQ ID NO: 25 shows the amino acid sequence of the heavy chain constant region, the nucleotide sequence of which is SEQ ID NO: 26; SEQ ID NO: 27 shows the amino acid sequence of the light chain constant region, the nucleotide sequence of which is SEQ ID NO: 28.

### Example 3. Construction of Light Chain of Novel CD20 Antibody

The nucleotide sequence of Rituximab light chain variable region (SEQ ID NO: 24) obtained in Example 1 and the nucleotide sequence of antibody light chain constant region (SEQ ID NO: 28) obtained in Example 2 were used as templates, and nucleotide sequence of the Rituximab light chain variable region was fused with that of the antibody light chain constant region using the method of overlap PCR, and then loaded into an expression vector. SEQ ID NO: 29 shows the amino acid sequence of novel CD20 antibody light chain, and the nucleotide sequence of which is SEQ ID NO:30.

### Example 4. Construction of Heavy Chain of Novel CD20 Antibody

The nucleotide sequence of novel CD20 antibody heavy chain variable region (SEQ ID NO: 4, 6, 8, 10, 12, 14, 16, 18, 20, 22) obtained in Example 1 and the nucleotide sequence of antibody heavy chain constant region (SEQ ID NO: 25) obtained in Example 2 were used as templates, and the nucleotide sequence of novel CD20 antibody heavy chain variable region was fused with that of the antibody heavy chain constant region using the method of overlap PCR, and then loaded into an expression vector. SEQ ID NO: 31 shows the amino acid sequence of novel CD20 antibody H102YH heavy chain, and the nucleotide sequence of which is SEQ ID NO:32. SEQ ID NO: 33 shows the amino acid sequence of novel CD20 antibody H102YL heavy chain, and the nucleotide sequence of which is SEQ ID NO:34. SEQ ID NO: 35 shows the amino acid sequence of novel CD20 antibody H102YM heavy chain, and the nucleotide sequence of which is SEQ ID NO:36. SEQ ID NO: 37 shows the amino acid sequence of novel CD20 antibody H102YN heavy chain, and the nucleotide sequence of which is SEQ ID NO:38. SEQ ID NO: 39 shows the amino acid sequence of novel CD20 antibody H102YQ heavy chain, and the nucleotide sequence of which is SEQ ID NO:40. SEQ ID NO: 41 shows the amino acid sequence of novel CD20 antibody H102YR heavy chain, and the nucleotide sequence of which is SEQ ID NO:42. SEQ ID NO: 43 shows the amino acid sequence of novel CD20 antibody H102YS heavy chain, and the nucleotide sequence of which is SEQ ID NO:44. SEQ ID NO: 45 shows the amino acid sequence of novel CD20 antibody H102YT heavy chain, and the nucleotide sequence of which is SEQ ID NO:46. SEQ ID NO: 47 shows the amino acid sequence of novel CD20 antibody H102YV heavy chain, and the nucleotide sequence of which is SEQ ID NO:48. SEQ ID NO: 49 shows the amino acid sequence of novel CD20 antibody H102YW heavy chain, and the nucleotide sequence of which is SEQ ID NO:50.

### Example 5. Expression and Purification of Novel CD20 Antibody

3×10⁵ CHO-K1 cells (ATCC CRL-9618) were seeded in 3.5 cm tissue culture dishes, and transfected when the cells were cultured to 90%-95% fusion: 10 μg plasmid of the novel CD20 antibody heavy chain (SEQ ID NO: 32, 34, 36, 38, 40, 42, 44, 46, 48, 50) together with 4 μg Rituximab light chain (SEQ ID NO: 30) and 20 μl of Lipofectamine 2000 Reagent (product from Invitrogen) were dissolved in 500 μl of serum-free DMEM medium respectively. After standing for 5 minutes at the room temperature, the above two liquids were mixed and incubated at room temperature for 20 minutes to form a DNA-liposome complex. During this time, the serum-containing medium in the culture dish was replaced with 3 ml of serum-free DMEM medium. Then the formed DNA-liposome complex was added to the plate. After 4 hours of incubation in a CO₂ incubator, 2 ml of DMEM complete medium containing 10% serum was added, and the culture was continued in a CO₂ incubator. After 24 h of transfection, the cell culture medium was changed into selection medium supplemented with 600 μg/ml G418 for screening resistant clones. Cell culture supernatants were screened for high-expression clones using ELISA: goat-anti-human IgG (Fc) was coated on ELISA plate, overnight at 4° C., and the plate was blocked with 2% BSA-PBS at 37° C. for 2 h. Then the supernatant or standard (Human myeloma IgG1, κ) of the resistant clones to be tested was added and the plate was incubated at 37° C. for 2 h. HRP-goat anti-human IgG (κ) was added for binding reaction, and the plate was incubated at 37° C. for 1 h. TMB was added and the plate was incubated at 37° C. for 5 min. And finally the reaction was terminated by H₂SO₄ and the A450 value was measured. The highly expressed clones obtained by the screening were expanded and cultured in a serum-free medium, and the novel CD20 antibody was isolated and purified using a Protein A affinity column (product from GE). The purified antibody was dialyzed in PBS, and finally the concentration of the purified antibody was quantitatively determined by ultraviolet absorption.

### Experiment 1. Novel CD20 Antibody Binding Activity Assay

All antibody avidities were determined using flow cytometry (Li B, Zhao L, Wang C, Guo H, Wu L, ZhangX, Qian W, Wang H, Guo Y. “The protein-protein interface evolution acts in a similar way to antibody affinity maturation.” J Biol Chem 2010; 285:3865-71.). Briefly, after diluted in equal concentration proportions, the purified fluorescently labeled antibody was incubated with the CD20 protein-positive lymphoma cell Raji (ATCC CCL-86) for 45 minutes on ice, and then washed twice and subjected to flow cytometry. The binding activity of the antibody was further evaluated based on the change in fluorescence intensity of the antibody after binding to the cells at different antibody concentrations. As shown in FIG. 1, these Rituximab mutants exhibited similar binding activities as the parental Rituximab antibody.

### Experiment 2. CDC Killing Activity of Novel CD20 Antibodies

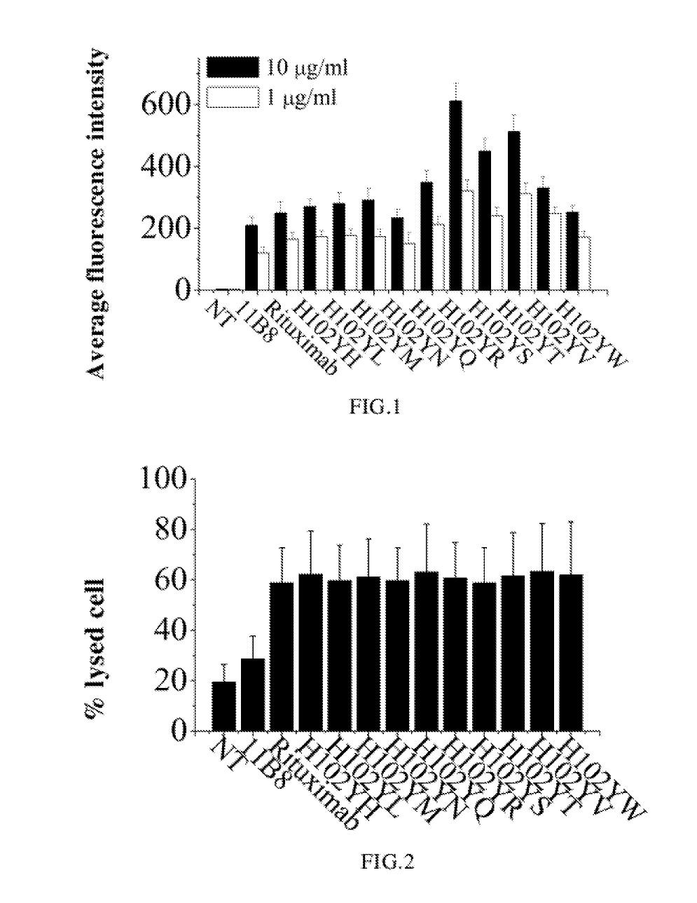

Raji cells (ATCC CCL-86), together with 11B8, Rituximab or the mutant thereof two times diluted starting with concentration of 10 μg/ml, was incubated in phenol red-free medium at 37° C. for 1 hour. Human serum was added at a volume ratio of 10%, and culture was continued at 37° C. for 4 hours, and then analyzed using a non-isotopic cytotoxicity test kit by a method provided in the product specification, to calculate the percentage of lysed cells. The treatment group in which no antibody was added was used as an experimental negative control. As shown in FIG. 2, both Rituximab and its mutants were able to induce intense CDC killing at the same degree.

### Experiment 3. Cell Death Inducing Activity of Novel CD20 Antibodies

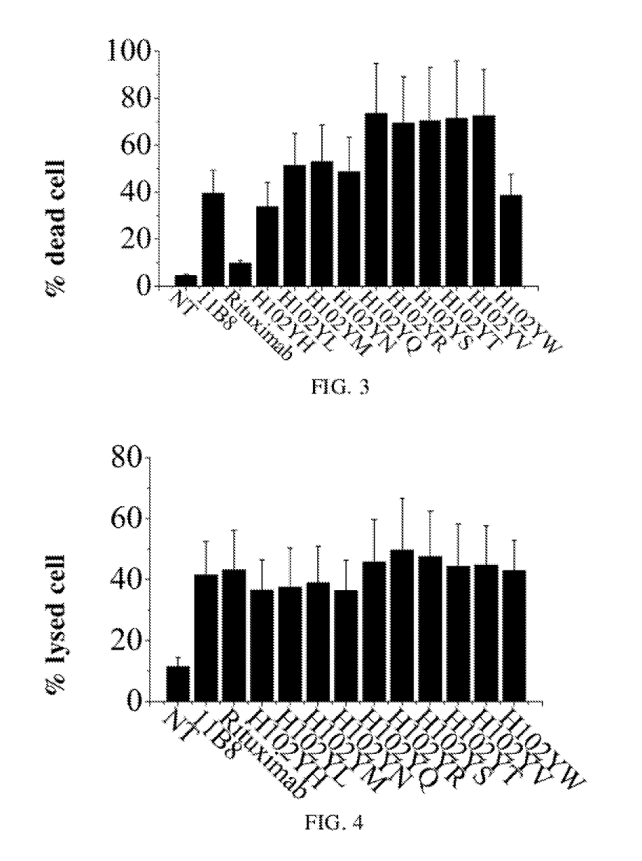

Raji cells (ATCC CCL-86), together with 11B8, Rituximab or the mutant thereof two times diluted starting with concentration of 10 μg/ml, was incubated at 37° C. for 48 h. Cells were washed twice, and then the percentage of cell death was measured using the Annexin V/PI kit (product from BD) according to the product specification. The treatment group in which no antibody was added was used as an experimental negative control, while treatment group treated with 11B8 was used as an experimental positive control. As shown in FIG. 3, type II CD20 antibody 11B8 was able to induce strong cell death, whereas type I CD20 antibody was unable to induce strong cell death. Although the parental antibody Rituximab was unable to induce strong cell death, several of these Rituximab mutants showed strong cell death-inducing ability.

### Experiment 4. ADCC Killing Activity of Novel CD20 Antibodies

Raji cells (ATCC CCL-86), together with different dilutions of 11B8, Rituximab and the mutants thereof, PBS or isotype control antibody, was incubated in phenol red-free medium for 1 hour at room temperature. Freshly isolated human peripheral blood mononuclear cells were added at an effector/target ratio of 50:1, and incubation was continued for 1 hour at 37° C., and finally CytoTox 96 non-isotopic cytotoxicity assay kit (Promega product) was used to analyze and calculate the percentage of lysed cells, according to the method provided in the product specification. The treatment groups treated with PBS and the isotype control antibody respectively was the experimental negative control group. As shown in FIG. 4, both type I and type II CD20 antibodies were able to induce intense ADCC killing.

### Experiment 5. Anti-Tumor Activity of Novel CD20 Antibodies in Mouse

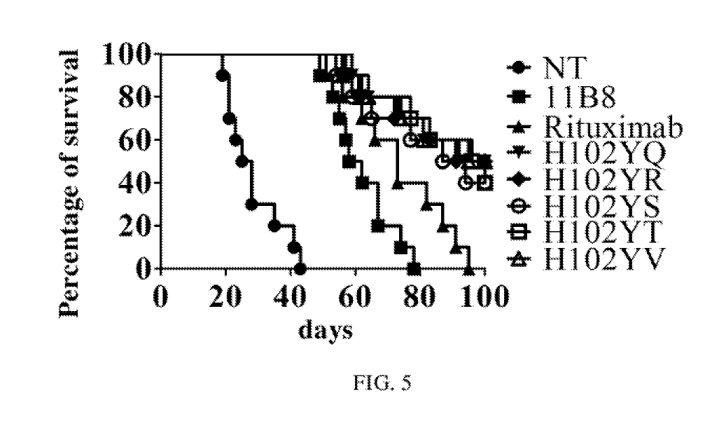

Six to eight week old of female SCID mice were selected, and then CD20-positive B lymphoma cells Raji (ATCC CCL-86) were inoculated into the tail vein at 0 day. After 5 days, tumor-bearing mice were tail vein injected with PBS or 10 μg, 100 μg, or 500 μg of 11B8, Rituximab, Rituximab mutant and irrelevant antibody control Her2 antibody Trastuzumab, respectively. SCID mice injected with PBS were grouped as an experimental control group. The changes of body weight and occurrence of limb paralysis in each group were observed regularly. The survival rate of mice was analyzed, and the anti-tumor efficacy of different antibodies was evaluated. As shown in FIG. 5, the novel CD20 antibody, which has the advantages of both type I and type II CD20 antibodies, exhibits a better anti-tumor activity than the parental type I CD20 antibody Rituximab and type II CD20 antibodies.

1-12. (canceled)

13. A CD20 antibody having both the advantages of type I and type II CD20 antibodies, wherein the antibody retains the strong CDC killing effect of the type I CD20 antibody Rituximab and the strong cell death effect of the type II CD20, and exhibits better anti-tumor activity than the type I CD20 antibody Rituximab or type II CD20 antibody 11B8;
wherein the antibody consists of four peptide chains, which are Rituximab heavy chain mutant and Rituximab light chain, respectively;
wherein the Rituximab heavy chain mutant is selected from the group consisting of:
102YH, 102YL, 102YM, 102YN, 102YQ, 102YR, 102YS, 102YT, 102YV, and 102YW.

14. The CD20 antibody as claim 13, wherein the antibody consists of a heavy chain mutant selected from the group consisting of:
Rituximab antibody heavy chain mutant H102YH, the amino acid sequence of the heavy chain variable region thereof is SEQ ID NO:3;
Rituximab antibody heavy chain mutant H102YL, the amino acid sequence of the heavy chain variable region thereof is SEQ ID NO:5;
Rituximab antibody heavy chain mutant H102YM, the amino acid sequence of the heavy chain variable region thereof is SEQ ID NO:7;
Rituximab antibody heavy chain mutant H102YN, the amino acid sequence of the heavy chain variable region thereof is SEQ ID NO:9;
Rituximab antibody heavy chain mutant H102YQ, the amino acid sequence of the heavy chain variable region thereof is SEQ ID NO:11;
Rituximab antibody heavy chain mutant H102YR, the amino acid sequence of the heavy chain variable region thereof is SEQ ID NO:13;
Rituximab antibody heavy chain mutant H102YS, the amino acid sequence of the heavy chain variable region thereof is SEQ ID NO:15;
Rituximab antibody heavy chain mutant H102YT, the amino acid sequence of the heavy chain variable region thereof is SEQ ID NO:17;
Rituximab antibody heavy chain mutant H102YV, the amino acid sequence of the heavy chain variable region thereof is SEQ ID NO:19; and
Rituximab antibody heavy chain mutant H102YW, the amino acid sequence of the heavy chain variable region thereof is SEQ ID NO:21;
and the CD20 antibody consists of the light chain of the Rituximab antibody, wherein the amino acid sequence of the light chain variable region thereof is SEQ ID NO: 23.

15. The antibody of claim 13, wherein the antibody consists of a heavy chain mutant selected from the group consisting of:
Rituximab antibody heavy chain mutant H102YH whose amino acid sequence is SEQ ID NO:31;
Rituximab antibody heavy chain mutant H102YL whose amino acid sequence is SEQ ID NO:33;
Rituximab antibody heavy chain mutant H102YM whose amino acid sequence is SEQ ID NO:35;
Rituximab antibody heavy chain mutant H102YN whose amino acid sequence is SEQ ID NO:37;
Rituximab antibody heavy chain mutant H102YQ whose amino acid sequence is SEQ ID NO:39;
Rituximab antibody heavy chain mutant H102YR whose amino acid sequence is SEQ ID NO:41;
Rituximab antibody heavy chain mutant H102YS whose amino acid sequence is SEQ ID NO:43;
Rituximab antibody heavy chain mutant H102YT whose amino acid sequence is SEQ ID NO:45;
Rituximab antibody heavy chain mutant H102YV whose amino acid sequence is SEQ ID NO:47; and
Rituximab antibody heavy chain mutant H102YW whose amino acid sequence is SEQ ID NO:49;
and the CD20 antibody consists of the light chain of the Rituximab antibody whose amino acid sequence is SEQ ID NO: 29.

16. The antibody of claim 15, wherein the antibody consists of a heavy chain mutant selected from the group consisting of:
Rituximab antibody heavy chain mutant H102YR whose amino acid sequence is SEQ ID NO:41;
Rituximab antibody heavy chain mutant H102YS whose amino acid sequence is SEQ ID NO:43;
Rituximab antibody heavy chain mutant H102YW whose amino acid sequence is SEQ ID NO:45;

17. An isolated nucleotide molecule encoding a CD20 antibody of claim 13.

18. The nucleotide molecule of claim 17, which is selected from the group consisting of:
(a) a nucleotide sequence encoding an antibody light chain selected from the group consisting of: SEQ ID No: 30; and
(b) a nucleotide sequence encoding an antibody heavy chain selected from the group consisting of: SEQ ID No: 32, 34, 36, 38, 40, 42, 44, 46, 48 and 50.

19. A method for treating tumor, comprising administering the CD20 antibody of claim 13.

20. The method of claim 19, further comprising administering other anti-tumor drugs.

21. A method of claim 19 wherein the CD20 antibody of claim 13 is formulated with a pharmaceutically acceptable carrier.

22. The method of claim 21, further comprising administering other anti-tumor drugs.


SEQUENCE LISTING (50 sequences)

SEQ ID NO: 1 - protein, 121 aa, Mus musculus [sequence omitted]

SEQ ID NO: 2 - DNA, 363 bp, Mus musculus
caggtacaactacagcagcctggggctgagctggtgaagcctggggcctcagtgaagatgtcctgcaaggcttctggctacacatttaccagttacaatatgcactgggtaaagcagacacctggtcggggcctggaatggattggagctatttatccaggaaatggtgatacttcctacaatcagaagttcaagggcaaggccacactgactgcagacaaatcctccagcacagcctacatgcagctcagcagcctgacatctgaagactctgcggtctattactgtgcaagatcgacttactacggcggtgactggtacttcaatgtctggggcgcagggaccacggtcaccgtctctgca

SEQ ID NO: 3 - protein, 121 aa, Mus musculus
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYHGGDWYFNVWGAGTTVTVSA

SEQ ID NO: 4 - DNA, 363 bp, Mus musculus
caggtacaactacagcagcctggggctgagctggtgaagcctggggcctcagtgaagatgtcctgcaaggcttctggctacacatttaccagttacaatatgcactgggtaaagcagacacctggtcggggcctggaatggattggagctatttatccaggaaatggtgatacttcctacaatcagaagttcaagggcaaggccacactgactgcagacaaatcctccagcacagcctacatgcagctcagcagcctgacatctgaagactctgcggtctattactgtgcaagatcgacttaccacggcggtgactggtacttcaatgtctggggcgcagggaccacggtcaccgtctctgca

SEQ ID NO: 5 - protein, 121 aa, Mus musculus
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYLGGDWYFNVWGAGTTVTVSA

SEQ ID NO: 6 - DNA, 363 bp, Mus musculus
caggtacaactacagcagcctggggctgagctggtgaagcctggggcctcagtgaagatgtcctgcaaggcttctggctacacatttaccagttacaatatgcactgggtaaagcagacacctggtcggggcctggaatggattggagctatttatccaggaaatggtgatacttcctacaatcagaagttcaagggcaaggccacactgactgcagacaaatcctccagcacagcctacatgcagctcagcagcctgacatctgaagactctgcggtctattactgtgcaagatcgacttacctcggcggtgactggtacttcaatgtctggggcgcagggaccacggtcaccgtctctgca

SEQ ID NO: 7 - protein, 121 aa, Mus musculus
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYMGGDWYFNVWGAGTTVTVSA

SEQ ID NO: 8 - DNA, 363 bp, Mus musculus
caggtacaactacagcagcctggggctgagctggtgaagcctggggcctcagtgaagatgtcctgcaaggcttctggctacacatttaccagttacaatatgcactgggtaaagcagacacctggtcggggcctggaatggattggagctatttatccaggaaatggtgatacttcctacaatcagaagttcaagggcaaggccacactgactgcagacaaatcctccagcacagcctacatgcagctcagcagcctgacatctgaagactctgcggtctattactgtgcaagatcgacttacatgggcggtgactggtacttcaatgtctggggcgcagggaccacggtcaccgtctctgca

SEQ ID NO: 9 - protein, 121 aa, Mus musculus
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYNGGDWYFNVWGAGTTVTVSA

SEQ ID NO: 10 - DNA, 363 bp, Mus musculus
caggtacaactacagcagcctggggctgagctggtgaagcctggggcctcagtgaagatgtcctgcaaggcttctggctacacatttaccagttacaatatgcactgggtaaagcagacacctggtcggggcctggaatggattggagctatttatccaggaaatggtgatacttcctacaatcagaagttcaagggcaaggccacactgactgcagacaaatcctccagcacagcctacatgcagctcagcagcctgacatctgaagactctgcggtctattactgtgcaagatcgacttacaacggcggtgactggtacttcaatgtctggggcgcagggaccacggtcaccgtctctgca

SEQ ID NO: 11 - protein, 121 aa, Mus musculus
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYQGGDWYFNVWGAGTTVTVSA

SEQ ID NO: 12 - DNA, 363 bp, Mus musculus
caggtacaactacagcagcctggggctgagctggtgaagcctggggcctcagtgaagatgtcctgcaaggcttctggctacacatttaccagttacaatatgcactgggtaaagcagacacctggtcggggcctggaatggattggagctatttatccaggaaatggtgatacttcctacaatcagaagttcaagggcaaggccacactgactgcagacaaatcctccagcacagcctacatgcagctcagcagcctgacatctgaagactctgcggtctattactgtgcaagatcgacttaccaaggcggtgactggtacttcaatgtctggggcgcagggaccacggtcaccgtctctgca

SEQ ID NO: 13 - protein, 121 aa, Mus musculus
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYRGGDWYFNVWGAGTTVTVSA

SEQ ID NO: 14 - DNA, 363 bp, Mus musculus
caggtacaactacagcagcctggggctgagctggtgaagcctggggcctcagtgaagatgtcctgcaaggcttctggctacacatttaccagttacaatatgcactgggtaaagcagacacctggtcggggcctggaatggattggagctatttatccaggaaatggtgatacttcctacaatcagaagttcaagggcaaggccacactgactgcagacaaatcctccagcacagcctacatgcagctcagcagcctgacatctgaagactctgcggtctattactgtgcaagatcgacttaccgcggcggtgactggtacttcaatgtctggggcgcagggaccacggtcaccgtctctgca

SEQ ID NO: 15 - protein, 121 aa, Mus musculus
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYSGGDWYFNVWGAGTTVTVSA

SEQ ID NO: 16 - DNA, 363 bp, Mus musculus
caggtacaactacagcagcctggggctgagctggtgaagcctggggcctcagtgaagatgtcctgcaaggcttctggctacacatttaccagttacaatatgcactgggtaaagcagacacctggtcggggcctggaatggattggagctatttatccaggaaatggtgatacttcctacaatcagaagttcaagggcaaggccacactgactgcagacaaatcctccagcacagcctacatgcagctcagcagcctgacatctgaagactctgcggtctattactgtgcaagatcgacttactccggcggtgactggtacttcaatgtctggggcgcagggaccacggtcaccgtctctgca

SEQ ID NO: 17 - protein, 121 aa, Mus musculus
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYTGGDWYFNVWGAGTTVTVSA

SEQ ID NO: 18 - DNA, 363 bp, Mus musculus
caggtacaactacagcagcctggggctgagctggtgaagcctggggcctcagtgaagatgtcctgcaaggcttctggctacacatttaccagttacaatatgcactgggtaaagcagacacctggtcggggcctggaatggattggagctatttatccaggaaatggtgatacttcctacaatcagaagttcaagggcaaggccacactgactgcagacaaatcctccagcacagcctacatgcagctcagcagcctgacatctgaagactctgcggtctattactgtgcaagatcgacttacaccggcggtgactggtacttcaatgtctggggcgcagggaccacggtcaccgtctctgca

SEQ ID NO: 19 - protein, 121 aa, Mus musculus
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYVGGDWYFNVWGAGTTVTVSA

SEQ ID NO: 20 - DNA, 363 bp, Mus musculus
caggtacaactacagcagcctggggctgagctggtgaagcctggggcctcagtgaagatgtcctgcaaggcttctggctacacatttaccagttacaatatgcactgggtaaagcagacacctggtcggggcctggaatggattggagctatttatccaggaaatggtgatacttcctacaatcagaagttcaagggcaaggccacactgactgcagacaaatcctccagcacagcctacatgcagctcagcagcctgacatctgaagactctgcggtctattactgtgcaagatcgacttacgtcggcggtgactggtacttcaatgtctggggcgcagggaccacggtcaccgtctctgca

SEQ ID NO: 21 - protein, 121 aa, Mus musculus
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYWGGDWYFNVWGAGTTVTVSA

SEQ ID NO: 22 - DNA, 363 bp, Mus musculus; identical to SEQ ID NO: 6

SEQ ID NO: 23 - protein, 106 aa, Mus musculus [sequence omitted]

SEQ ID NO: 24 - DNA, 318 bp, Mus musculus
caaattgttctctcccagtctccagcaatcctgtctgcatctccaggggagaaggtcacaatgacttgcagggccagctcaagtgtaagttacatccactggttccagcagaagccaggatcctcccccaaaccctggatttatgccacatccaacctggcttctggagtccctgttcgcttcagtggcagtgggtctgggacctcttactctctcacaatcagtagagtggaggctgaagatgctgccacttattactgccagcagtggactagtaacccacccacgttcggtggtgggaccaagctggagatcaaa

SEQ ID NO: 25 - protein, 330 aa, Homo sapiens [sequence omitted]

SEQ ID NO: 26 - DNA, 993 bp, Homo sapiens
gcctccaccaagggcccatcggtcttccccctggcaccctcctccaagagcacctctgggggcacagcggccctgggctgcctggtcaaggactacttccccgaaccggtgacggtgtcttggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggtaaatga

SEQ ID NO: 27 - protein, 105 aa, Homo sapiens [sequence omitted]

SEQ ID NO: 28 - DNA, 318 bp, Homo sapiens
gtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 29 - protein, 211 aa, Homo sapiens
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - DNA, 636 bp, Homo sapiens
caaattgttctctcccagtctccagcaatcctgtctgcatctccaggggagaaggtcacaatgacttgcagggccagctcaagtgtaagttacatccactggttccagcagaagccaggatcctcccccaaaccctggatttatgccacatccaacctggcttctggagtccctgttcgcttcagtggcagtgggtctgggacctcttactctctcacaatcagtagagtggaggctgaagatgctgccacttattactgccagcagtggactagtaacccacccacgttcggtggtgggaccaagctggagatcaaagtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 31 - protein, 451 aa, Homo sapiens
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYHGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - DNA, 1356 bp, Homo sapiens
caggtacaactacagcagcctggggctgagctggtgaagcctggggcctcagtgaagatgtcctgcaaggcttctggctacacatttaccagttacaatatgcactgggtaaagcagacacctggtcggggcctggaatggattggagctatttatccaggaaatggtgatacttcctacaatcagaagttcaagggcaaggccacactgactgcagacaaatcctccagcacagcctacatgcagctcagcagcctgacatctgaagactctgcggtctattactgtgcaagatcgacttaccacggcggtgactggtacttcaatgtctggggcgcagggaccacggtcaccgtctctgcagcctccaccaagggcccatcggtcttccccctggcaccctcctccaagagcacctctgggggcacagcggccctgggctgcctggtcaaggactacttccccgaaccggtgacggtgtcttggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggtaaatga

SEQ ID NO: 33 - protein, 451 aa, Homo sapiens
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYLGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - DNA, 1356 bp, Homo sapiens
caggtacaactacagcagcctggggctgagctggtgaagcctggggcctcagtgaagatgtcctgcaaggcttctggctacacatttaccagttacaatatgcactgggtaaagcagacacctggtcggggcctggaatggattggagctatttatccaggaaatggtgatacttcctacaatcagaagttcaagggcaaggccacactgactgcagacaaatcctccagcacagcctacatgcagctcagcagcctgacatctgaagactctgcggtctattactgtgcaagatcgacttacctcggcggtgactggtacttcaatgtctggggcgcagggaccacggtcaccgtctctgcagcctccaccaagggcccatcggtcttccccctggcaccctcctccaagagcacctctgggggcacagcggccctgggctgcctggtcaaggactacttccccgaaccggtgacggtgtcttggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggtaaatga

SEQ ID NO: 35 - protein, 451 aa, Homo sapiens
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYMGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - DNA, 1356 bp, Homo sapiens
caggtacaactacagcagcctggggctgagctggtgaagcctggggcctcagtgaagatgtcctgcaaggcttctggctacacatttaccagttacaatatgcactgggtaaagcagacacctggtcggggcctggaatggattggagctatttatccaggaaatggtgatacttcctacaatcagaagttcaagggcaaggccacactgactgcagacaaatcctccagcacagcctacatgcagctcagcagcctgacatctgaagactctgcggtctattactgtgcaagatcgacttacatgggcggtgactggtacttcaatgtctggggcgcagggaccacggtcaccgtctctgcagcctccaccaagggcccatcggtcttccccctggcaccctcctccaagagcacctctgggggcacagcggccctgggctgcctggtcaaggactacttccccgaaccggtgacggtgtcttggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggtaaatga

SEQ ID NO: 37 - protein, 451 aa, Homo sapiens
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYNGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - DNA, 1356 bp, Homo sapiens
caggtacaactacagcagcctggggctgagctggtgaagcctggggcctcagtgaagatgtcctgcaaggcttctggctacacatttaccagttacaatatgcactgggtaaagcagacacctggtcggggcctggaatggattggagctatttatccaggaaatggtgatacttcctacaatcagaagttcaagggcaaggccacactgactgcagacaaatcctccagcacagcctacatgcagctcagcagcctgacatctgaagactctgcggtctattactgtgcaagatcgacttacaacggcggtgactggtacttcaatgtctggggcgcagggaccacggtcaccgtctctgcagcctccaccaagggcccatcggtcttccccctggcaccctcctccaagagcacctctgggggcacagcggccctgggctgcctggtcaaggactacttccccgaaccggtgacggtgtcttggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggtaaatga

SEQ ID NO: 39 - protein, 451 aa, Homo sapiens
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYQGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - DNA, 1356 bp, Homo sapiens
caggtacaactacagcagcctggggctgagctggtgaagcctggggcctcagtgaagatgtcctgcaaggcttctggctacacatttaccagttacaatatgcactgggtaaagcagacacctggtcggggcctggaatggattggagctatttatccaggaaatggtgatacttcctacaatcagaagttcaagggcaaggccacactgactgcagacaaatcctccagcacagcctacatgcagctcagcagcctgacatctgaagactctgcggtctattactgtgcaagatcgacttaccaaggcggtgactggtacttcaatgtctggggcgcagggaccacggtcaccgtctctgcagcctccaccaagggcccatcggtcttccccctggcaccctcctccaagagcacctctgggggcacagcggccctgggctgcctggtcaaggactacttccccgaaccggtgacggtgtcttggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggtaaatga

SEQ ID NO: 41 - protein, 451 aa, Homo sapiens
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYRGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - DNA, 1356 bp, Homo sapiens
caggtacaactacagcagcctggggctgagctggtgaagcctggggcctcagtgaagatgtcctgcaaggcttctggctacacatttaccagttacaatatgcactgggtaaagcagacacctggtcggggcctggaatggattggagctatttatccaggaaatggtgatacttcctacaatcagaagttcaagggcaaggccacactgactgcagacaaatcctccagcacagcctacatgcagctcagcagcctgacatctgaagactctgcggtctattactgtgcaagatcgacttaccgcggcggtgactggtacttcaatgtctggggcgcagggaccacggtcaccgtctctgcagcctccaccaagggcccatcggtcttccccctggcaccctcctccaagagcacctctgggggcacagcggccctgggctgcctggtcaaggactacttccccgaaccggtgacggtgtcttggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggtaaatga

SEQ ID NO: 43 - protein, 451 aa, Homo sapiens
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYSGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - DNA, 1356 bp, Homo sapiens
caggtacaactacagcagcctggggctgagctggtgaagcctggggcctcagtgaagatgtcctgcaaggcttctggctacacatttaccagttacaatatgcactgggtaaagcagacacctggtcggggcctggaatggattggagctatttatccaggaaatggtgatacttcctacaatcagaagttcaagggcaaggccacactgactgcagacaaatcctccagcacagcctacatgcagctcagcagcctgacatctgaagactctgcggtctattactgtgcaagatcgacttactccggcggtgactggtacttcaatgtctggggcgcagggaccacggtcaccgtctctgcagcctccaccaagggcccatcggtcttccccctggcaccctcctccaagagcacctctgggggcacagcggccctgggctgcctggtcaaggactacttccccgaaccggtgacggtgtcttggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggtaaatga

SEQ ID NO: 45 - protein, 451 aa, Homo sapiens
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYTGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - DNA, 1356 bp, Homo sapiens
caggtacaactacagcagcctggggctgagctggtgaagcctggggcctcagtgaagatgtcctgcaaggcttctggctacacatttaccagttacaatatgcactgggtaaagcagacacctggtcggggcctggaatggattggagctatttatccaggaaatggtgatacttcctacaatcagaagttcaagggcaaggccacactgactgcagacaaatcctccagcacagcctacatgcagctcagcagcctgacatctgaagactctgcggtctattactgtgcaagatcgacttacaccggcggtgactggtacttcaatgtctggggcgcagggaccacggtcaccgtctctgcagcctccaccaagggcccatcggtcttccccctggcaccctcctccaagagcacctctgggggcacagcggccctgggctgcctggtcaaggactacttccccgaaccggtgacggtgtcttggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggtaaatga

SEQ ID NO: 47 - protein, 451 aa, Homo sapiens
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYVGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - DNA, 1356 bp, Homo sapiens
caggtacaactacagcagcctggggctgagctggtgaagcctggggcctcagtgaagatgtcctgcaaggcttctggctacacatttaccagttacaatatgcactgggtaaagcagacacctggtcggggcctggaatggattggagctatttatccaggaaatggtgatacttcctacaatcagaagttcaagggcaaggccacactgactgcagacaaatcctccagcacagcctacatgcagctcagcagcctgacatctgaagactctgcggtctattactgtgcaagatcgacttacgtcggcggtgactggtacttcaatgtctggggcgcagggaccacggtcaccgtctctgcagcctccaccaagggcccatcggtcttccccctggcaccctcctccaagagcacctctgggggcacagcggccctgggctgcctggtcaaggactacttccccgaaccggtgacggtgtcttggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggtaaatga

SEQ ID NO: 49 - protein, 451 aa, Homo sapiens
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYWGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - DNA, 1356 bp, Homo sapiens
caggtacaactacagcagcctggggctgagctggtgaagcctggggcctcagtgaagatgtcctgcaaggcttctggctacacatttaccagttacaatatgcactgggtaaagcagacacctggtcggggcctggaatggattggagctatttatccaggaaatggtgatacttcctacaatcagaagttcaagggcaaggccacactgactgcagacaaatcctccagcacagcctacatgcagctcagcagcctgacatctgaagactctgcggtctattactgtgcaagatcgacttactggggcggtgactggtacttcaatgtctggggcgcagggaccacggtcaccgtctctgcagcctccaccaagggcccatcggtcttccccctggcaccctcctccaagagcacctctgggggcacagcggccctgggctgcctggtcaaggactacttccccgaaccggtgacggtgtcttggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggtaaatga